(19) United States
(12) Patent Application Publication
(10) Pub. No.: US 2015/0071909 A1
(43) Pub. Date: Mar. 12, 2015

(54) METHODS AND COMPOSITIONS FOR REDUCING THE INCIDENCE OF POST-SURGICAL ADHESIONS

(71) Applicant: Saint Louis University, St. Louis, MO (US)

(72) Inventors: Alireza R. Rezaie, Eureka, MO (US); Peyman Dinarvand, St. Louis, MO (US)

(21) Appl. No.: 14/481,859

(22) Filed: Sep. 9, 2014

Related U.S. Application Data
(60) Provisional application No. 61/876,862, filed on Sep. 12, 2013.

(51) Int. Cl. A61K 38/48

(52) U.S. Cl. CPC A61K 38/4866
USPC 424/94.64

(57) ABSTRACT

The disclosure relates to a method of reducing the incidence of post-surgical adhesions in a subject undergoing surgery. More specifically, the method relates to reducing the incidence of post-surgical adhesions with the topically administration of variants of activated protein C with cytoprotective and antiinflammatory activity to the internal organs and tissues exposed and/or manipulated during surgery. The disclosure is also related to reducing the incidence of post-surgical adhesions with the topically administration of a variant of activated protein C with cytoprotective activity and reduced or no anticoagulant activity.

### CROSS REFERENCE TO RELATED APPLICATIONS

This application claims priority to provisional application 61/876,862, filed Sep. 12, 2013, which is hereby incorporated by reference in its entirety.

### GOVERNMENT FUNDING

This work was supported by National Institutes of Health grant no. HL 101917. The government of the United States has certain rights in this invention.

### FIELD OF THE INVENTION

The invention relates to methods and compositions, for reducing the incidence of post-surgical adhesion band formation resulting from general, abdominal, pelvic or cardiac surgery.

### BACKGROUND

Post-surgical adhesions are pathological fibrous bands which form between organs and tissues after surgery. Post-surgical adhesions due to abdominopelvic surgery, also known as peritoneal adhesions, are pathological fibrous bands which join intra-abdomen and intra-pelvic organs to each other and to peritoneal wall. Post-surgical adhesions may increase in strength with time, sometimes causing problems years after surgery. Post-surgical adhesions are also associated with high mortality and morbidity including intestinal obstruction, pelvic pain, and female infertility. Post-surgical adhesions are the most frequent result of abdominopelvic and cardiac surgery. Anyone undergoing abdominopelvic or cardiac surgery runs the risk of developing post-surgical adhesions; however, the risk is greater after operations on the lower abdomen and pelvis, including bowel and gynecological surgeries. Adhesion bands occur in 67% to 93% of patients undergoing general surgical operations and up to 97% of patients undergoing open gynecological pelvic surgeries (Liakakos et al, (2001) Dig Surg, 18:260-73; Menzies (1990) Ann R Coll Surg Engl, 72:60-3). Post-surgical adhesions are the most important causes of intestinal obstruction (Menzies (1992) Surgery annual, 24:27), pelvic pain (Sulaiman et al., (2000) The Journal of Pathology, 192:396-403), female infertility (Kavic (2002) JSLS, 6:99-109) and adhesion of heart to surrounding tissues (Seeger et al., (1997) Journal of Surgical Research, 68:63-66). Post-surgical adhesions are believed to be more common after surgical trauma, including mild surgical trauma of internal tissues and organs. Surgical trauma may include incisions, manipulations, surface desiccation, or the contact of the internal tissues with foreign materials, such as gauze, surgical gloves, or stitches. Attempts to reduce post-surgical adhesions through gentle tissue handling, meticulous hemostasis, or avoiding desiccation and contamination of the abdominal or pelvic cavity with foreign materials through the use of starch-free gloves, lint-free gauze and absorbable sutures have been attempted (Holmdahl et al. (1997) Eur J Surg Suppl, 56-62). However it is believed that superior surgical technique may only provide marginal improvement. The focus is now to develop methods and compositions which may be administrated at the time of surgery.

Anti-inflammatory agents, antibiotics, fibrinolytic agents and solid barriers have been used for the prevention of post-surgical adhesions (Hemadeh et al., (1993) Surgery, 114:907-10; Hellebrekers et al., (2000) Hum Reprod, 15:1358-63; Kusunoki et al., (2005) Surg Today, 35:940-5; Becker et al., (1996) J Am Coll Surg, 183:297-306; Dinarvand et al., (2012) J Surg Res, 172:e1-9); (Seeger et al., (1997) Journal of Surgical Research, 68:63-66). For example, inflammation may be reduced by the administration of drugs such as corticosteroids and non-steroidal anti-inflammatory drugs. The removal of fibrin deposits has been investigated using proteolytic and fibrinolytic enzymes, but these results have been unsatisfactory. Most successful have been the use of physical barriers, which physically prevent adjacent tissues from contacting each other and thereby reducing the probability that band formation will occur. Examples of barrier materials include films such as those formed from oxidized regenerated cellulose (e.g., Interceed™, Gynecare, Ethicon division of Johnson and Johnson, Arlington, Tex., USA), hyaluronate/carboxymethylcellulose (Seprafilm™, Genzyme Corporation, Cambridge, Mass.) and polytetrafluoroethylene (Preclude™, W.L. Gore & Associates, Flagstaff, Ariz., USA), among others. Selected agents including sodium hyaluronate/carboxymethylcellulose (HA/CMC) (Seprafilm) and oxidized regenerated cellulose (Interceed) have been approved by the FDA and are the gold standards for the prevention of adhesion bands. However, a limitation of using physical barriers is their site-specific nature, which requires the surgeon to predict where adhesions may occur, and to place these barriers accordingly.

There has been no satisfactory method of treatment for post-surgical adhesions. There exists a long felt need for a treatment that is effective and easy to apply. The Inventors have discovered that the topical administration of a low dose of activated protein C (APC) to the internal organs and surrounding tissues of the peritoneal, after the primary surgery or surgical manipulation is performed, but prior to closing of the major abdominopelvic incision, will produce a significantly better result than the currently accepted methods such as the use of Seprafilm for prevention of post-surgical adhesions. It is also disclosed herein that a variant of APC lacking anticoagulant activity but retaining cytoprotective activity is effective in preventing postsurgical adhesions. It is believed that this method may be applied generally to all surgeries where post-surgical adhesions occur.

### SUMMARY OF THE INVENTION

A method of reducing the incidence of post-surgical adhesions in a subject undergoing surgery, the method comprising, topically administering an effective amount of a variant of activated protein C which possesses cytoprotective activity to the internal organs and tissues exposed by surgery or subjected to surgical manipulation, prior to closing the major incision. In one embodiment, the method is used to apply a variant of activated protein C which possesses cytoprotective and antiinflammatory activity but possesses reduced or no anticoagulant activity.

### REFERENCE TO COLOR FIGURES

The application file contains at least one figure executed in color. Copies of this patent application publication with color photographs will be provided by the Office upon request and payment of the necessary fee.

### DESCRIPTION OF THE FIGURES

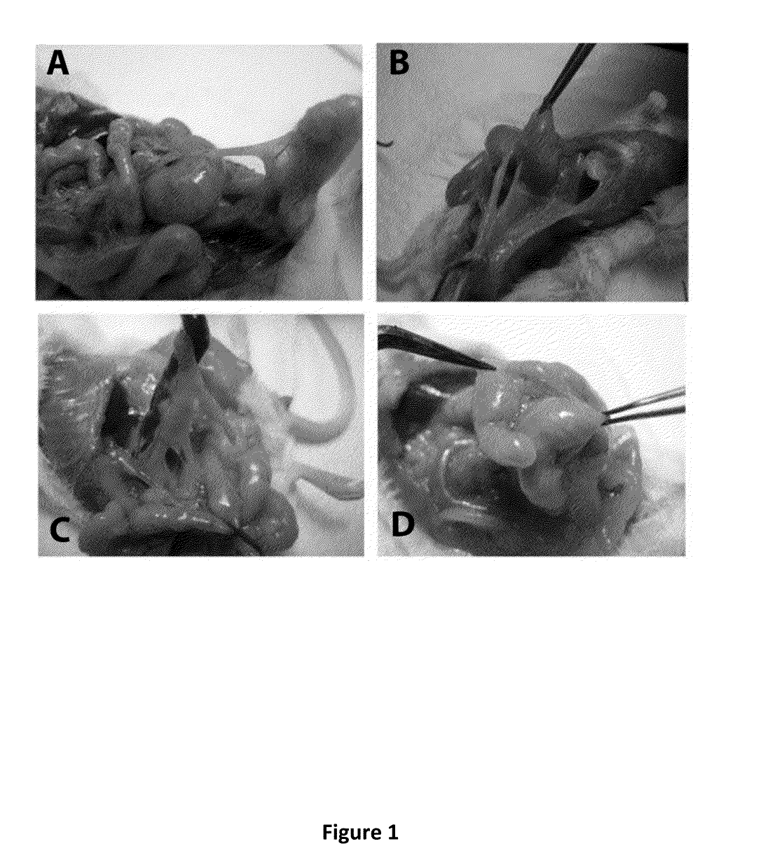

FIG. 1 illustrates intraperitoneal adhesion formation in mice. (A) Grade 1, only one band of adhesion between one viscera and the abdominal wall (Nair et al scale). A filmy thickness, Avascular bands (Leach et al scale). (B) Grade 2, two adhesion bands from viscera to abdominal wall (Nair et al scale). Limited vascularity, moderate thickness (Leach et al). (C) Grade 3, More than two bands between viscera or viscera to abdominal wall, or whole of intestines forming a mass without adherent to abdominal wall (Nair et al scale). (D) Grade 4, viscera directly adherent to abdominal wall (Nair et al). Grade 3, well vascularized, dense thickness (Leach et al scale).

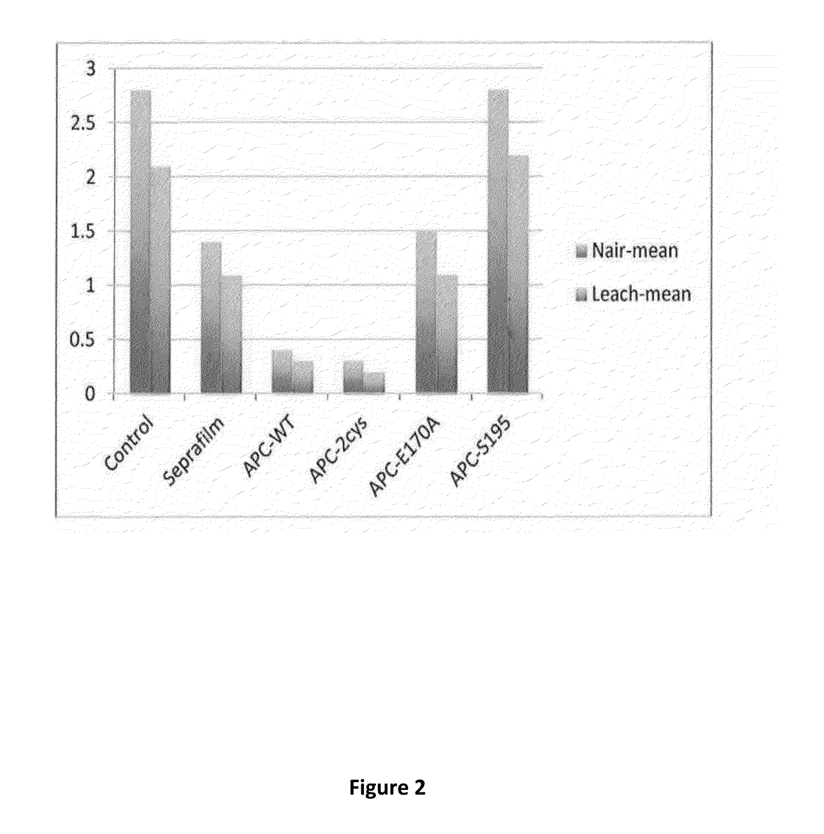

FIG. 2 shows adhesion scores of Control (saline solution), Seprafilm, APC wild-type, APC-2Cys (an APC variant possessing cytoprotective and antiinflammatory activity, but lacking anticoagulant activity), APC-E170A (an APC variant lacking cytoprotective and antiinflammatory activity, but possessing normal anticoagulant activity), and APC-S195A (an APC variant possessing neither cytoprotective and antiinflammatory activity nor anticoagulant activity), groups as explained by Nair et al; and Leach et al.

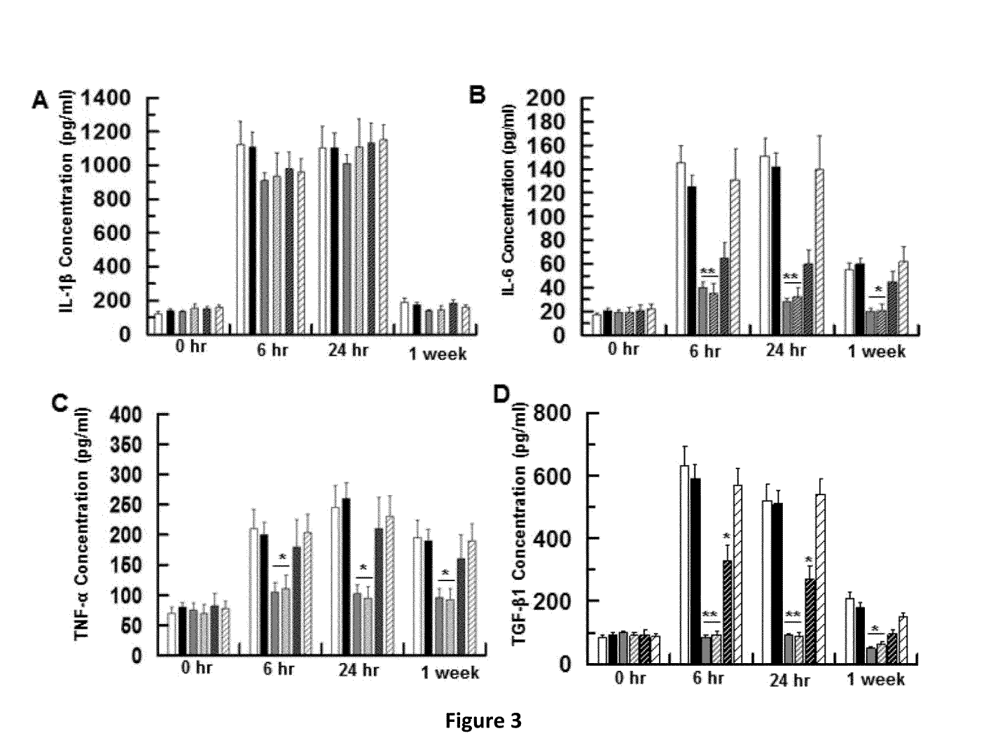

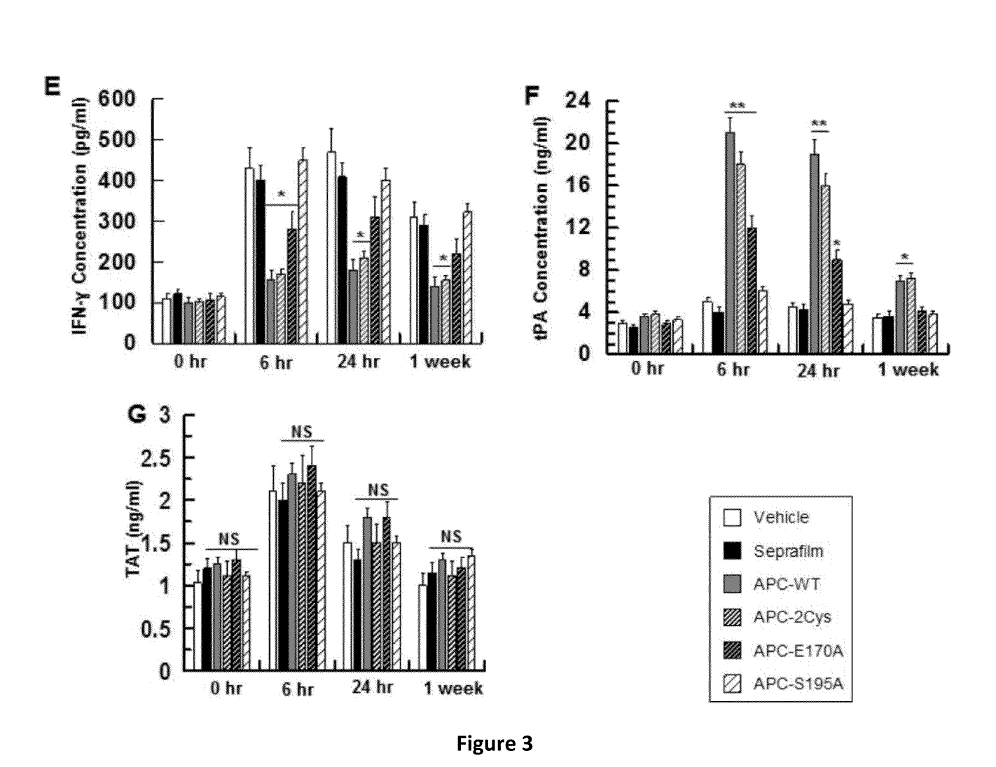

FIG. 3. Shows the analysis of peritoneal fluid concentrations of cytokines as well as tPA and plasma TAT complex in different animal groups. The concentrations of IL-1β (A), IL-6 (B), TNF-α (C), TGF-β1 (D), IFN-γ (E), tPA (F) and TAT complex (G) for all groups were measured by ELISA assays. The data are shown as mean±SD. *p<0.05 and **p<0.01 as compared to saline treated vehicle at each time point.

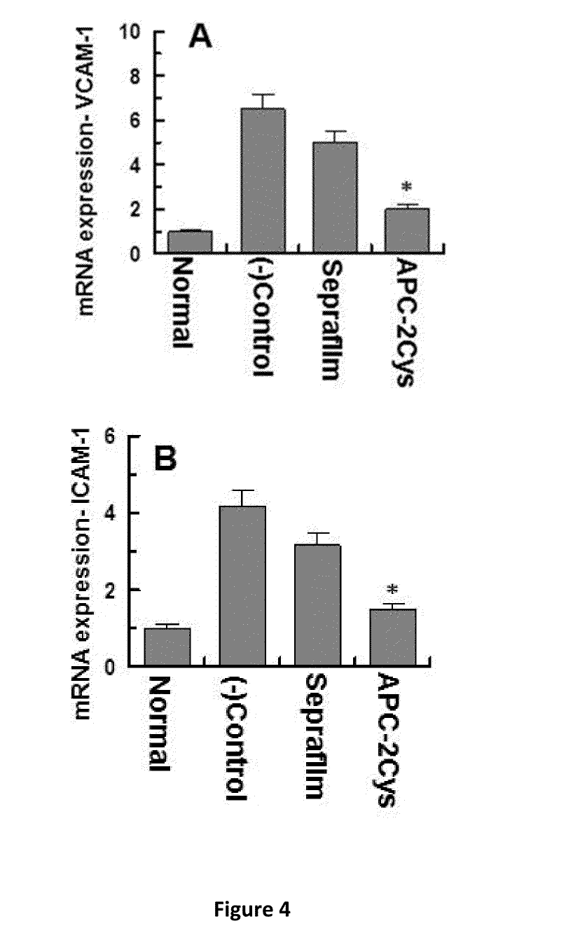

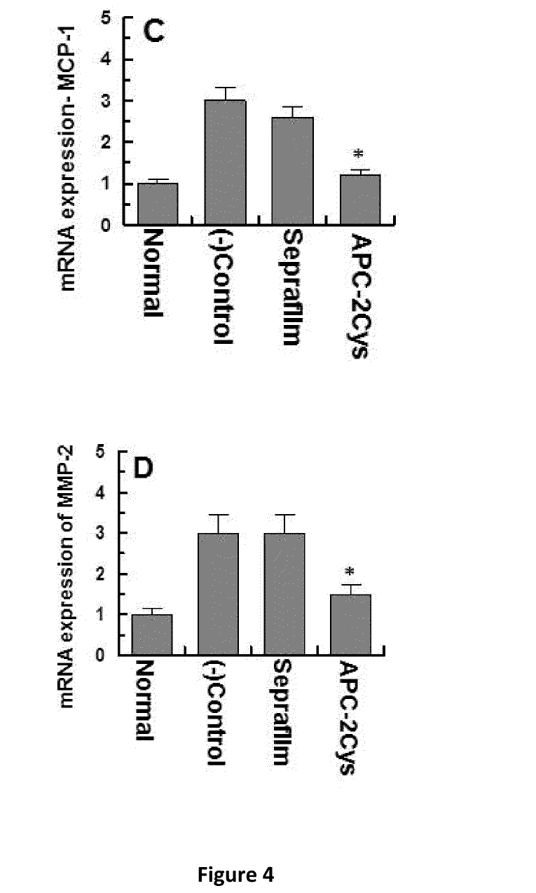

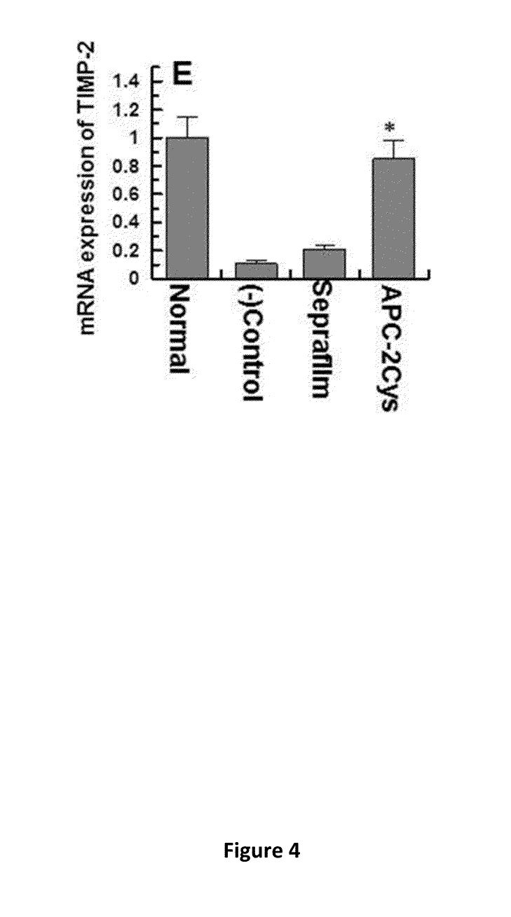

FIG. 4 shows the messenger RNA (mRNA) expression of different molecules derived from the peritoneal tissues of in different groups. (A) The mRNA expression of vascular cell adhesion molecule 1 (VCAM-1) was measured for APC-2Cys and Seprafilm treated groups. The term “Normal” in the figure represents the control group which has not undergone surgery. (−)Control represents mice undergone surgery to induce adhesion but not treated with either Seprafilm or APC. (B) The same as panel A except that the mRNA expression of intracellular adhesion molecule 1 (ICAM-1) was monitored. (C) The same as panel A except that the mRNA expression of monocyte chemotactic protein 1 (MCP-1) was measured. (D) The same as panel A except that the mRNA expression of matrix metalloproteinase 2 (MMP-2) was measured. (E) The same as panel A except that the mRNA expression of tissue inhibitor of metalloproteinase 2 (TIMP-2) was measured. All results are shown as mean±SD. The significant difference (P<0.05) has been shown between the APC-treated groups with (*) and the groups of Seprafilm and control.

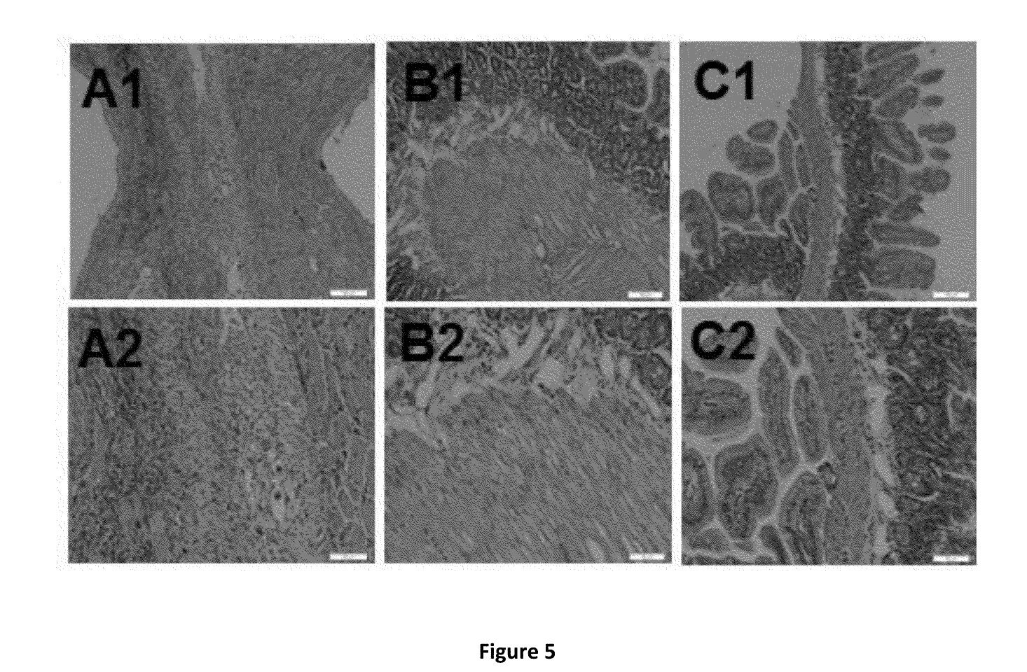

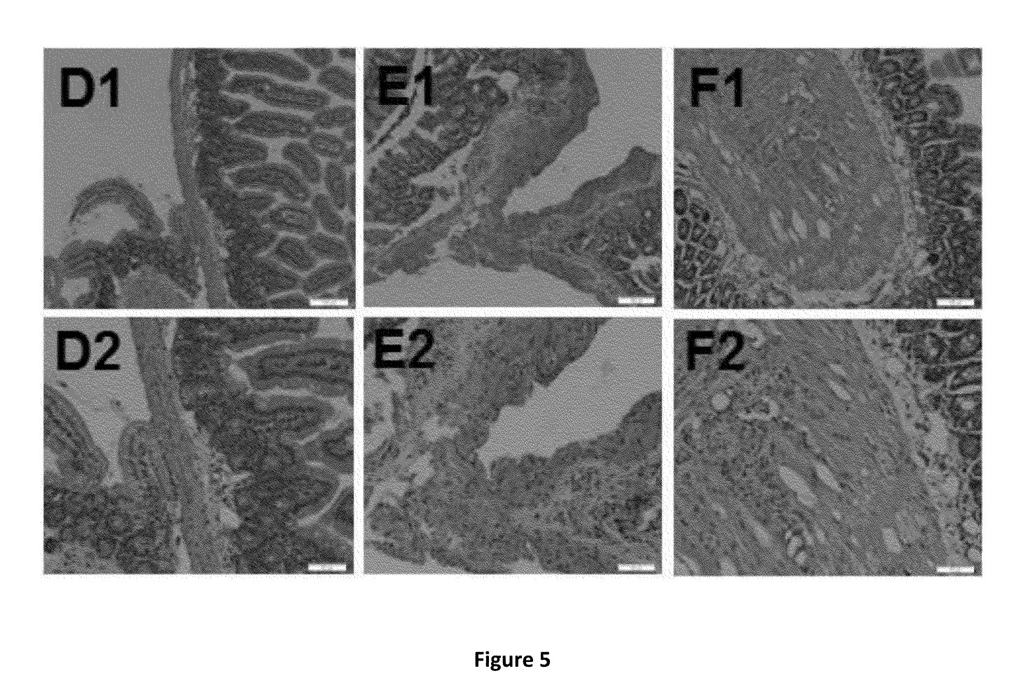

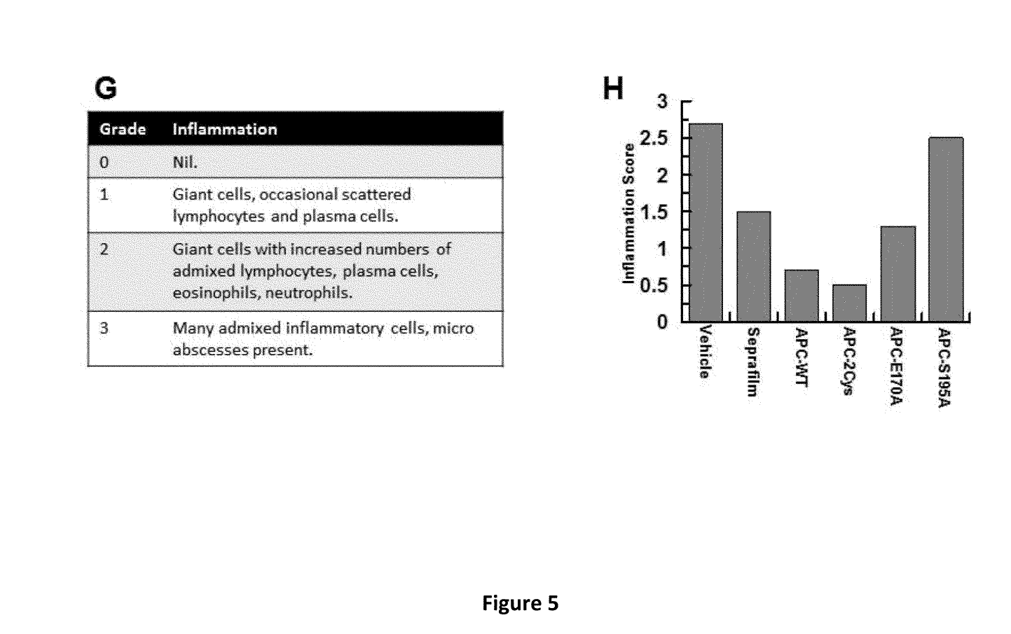

FIG. 5 Peritoneal tissue histology in two different magnifications. Representative images are derived from saline-treated control group (A1,A2), Seprafilm group (B1,B2), APC-WT group (C1,C2), APC-2Cys group (D1,D2), APC-E170A group (E1,E2) and APC-S195A (F1,F2). The numerical scoring of inflammation based on histology (G) is shown in panel H.

### DETAILED DESCRIPTION OF THE INVENTION

The invention provides a novel approach for preventing, inhibiting, treating, or reducing the incidence of, or reducing the risk of, post-surgical adhesions that may form between internal organs and tissues following general, abdominal, pelvic, abdominopelvic, or cardiac surgery.

Without wishing to be bound by theory, it is believed that post-surgical adhesions begin to form within hours or days after surgery. These may arise from mild surgical trauma such as a local abrasion, incision, or even the temporary drying of the surface of some organs or tissues, which may occur despite the surgeon's best efforts. Even mild trauma may result in inflammation which may release various inflammatory factors and/or cytokines. This may in turn cause increased vascular permeability, fibrinogen activation, and fibrin deposition. A fibrin matrix may become established between abutting organs and tissues, and the lining of the abdominal, pelvic, or chest cavity. The fibrin matrix may in turn become infiltrated with fibroblasts, which may in turn deposit collagen into the matrix, thereby fortifying the matrix and possibly inducing angiogenesis.

The Inventors reasoned that if this cascade of events can be reduced or prevented within the first few hours or days following surgery then subsequent matrix and adhesion band formation may be reduced or eliminated. The Inventors reasoned that activated protein C (APC), a natural anticoagulant and anti-inflammatory serine protease found in plasma may be effective if applied locally to the organs and tissues of the abdominal, pelvic, or chest cavity, following the surgical events that may result in trauma, in order to prevent these early stages of post-surgical adhesion formation.

The Inventors first reasoned that a topical application of APC intraperitoneally, or on sites of surgical trauma, may inhibit inflammatory events, the deposition of fibrin, or both, and reduce the incident of post-surgical band formation at a later date. To this end the Inventors devised a treatment method whereby APC (wild type APC) is applied topically to organs exposed through surgery, as well as generally to the abdominal, pelvic, or chest cavity, prior to closing of the major abdominal, pelvic, or chest incision. The topical application of APC, directly to sites with the potential for inflammation and fibrin deposition will also reduce the total amount of APC administered and possibly reduce collateral effects, including an increased incidence of bleeding that may be caused by APC when administered systemically. The most preferred effective amount of 50 micrograms of APC, per kilogram body weight of the subject, is relatively low compared to dosages that have been used systemically in the treatment of sepsis.

The Inventors have previously disclosed the use of wild type APC to reduce the incidence of post-surgical adhesions (see U.S. application Ser. No. 13/854,117 and PCT/US13/34750, incorporated herein by reference in their entirety). The Inventors disclose herein, the identification of the pertinent structural and functional properties of APC responsible for the observed reduction in post-surgical adhesions, as well as the use of variants of APC with retain cytoprotective properties, such as the anti-inflammatory and anti-apoptotic properties of APC, for reducing the incidence of post-surgical adhesions. One such variant designated APC-2Cys, retains cytoprotective activity, but demonstrates little or no anticoagulant activity. Activated protein C is well known for its cytoprotective and anticoagulative properties. To determine if either cytoprotective or anticoagulative activity was associated with the reduction in post-surgical adhesions; the Inventors tested genetically engineered variants of APC that were selectively deficient in these functional properties. The Inventors made the surprising discovery that a variant of APC which retained its cytoprotective activity but lacked anticoagulant activity was as effective as wild-type APC in reducing post-surgical adhesions.

It was previously shown that by replacing amino acid residues at position 264 and also position 279 of wild-type APC with cysteines it was possible to produce an APC mutant or variant that would form a disulfide bound between the substituted positions. The result was to form a crosslink in the 70-80 calcium binding loop of the polypeptide, which contributes to anticoagulant activity. This produced a variant of APC which retained normal cytoprotective activity but little or no anticoagulant activity, designated herein as activated protein C-2Cys or APC-2Cys (See U.S. Pat. No. 7,785,857). Using this variant of APC, the Inventors applied the methods described and demonstrated in for wild type APC (see U.S. application Ser. No. 13/854,117, hereby incorporated by reference in its entirety) to assess the ability of APC-2Cys to reduce post-surgical adhesions. As seen in the examples below, the Inventors found that APC-2 Cys was as effective as wild type APC at reducing the incidence of post-surgical adhesions. These results indicate that the anticoagulant activity, or the serine protease activity of APC, is not required for reducing post-surgical adhesions. The Inventors further confirmed this discovery by testing other genetically engineered APC variants. One such APC variant, designated APC-E170A, possessed anticoagulant activity, but lacked cytoprotective activity. When tested, APC-E170A was found to have poor anti-adhesive activity. Another variant, designated APC-S195A, possessed neither anticoagulant nor cytoprotective activity. APC-S195A also demonstrated poor anti-adhesive activity. The results indicate that the cytoprotective activity of APC is responsible for reducing the incident of post-surgical adhesions and that anticoagulant activity is not required. Therefore it is anticipated by the Inventors that the disclosed method may be used effectively for reducing the incident of post-surgical adhesions with any variant of APC that retains cytoprotective activity. A variant of activated protein C, designated activated protein C-2Cys, is disclosed herein as a non-limiting example of an APC variant with cytoprotective activity. Activated protein C-2Cys is also a non-limiting example of a variant of APC with cytoprotective activity and reduced anticoagulant activity. An APC variant with reduced anticoagulant activity, by way of example APC-2Cys, also provides the advantage in that it will not increase the incidence of bleeding as sometimes seen with the systemic administration of wild-type APC. Other non-limiting examples of APC variants with reduced anti-coagulant activity but normal cytoprotective activity are described by Griffin et al., in U.S. Pat. No. 7,498,305, (incorporated herein by reference in its entirely), which are anticipated to be effective in reducing the incidence of post-surgical adhesions.

One embodiment of the invention is a method of reducing the incidence of post-surgical adhesions by bathing the internal organs and tissues in an adequate volume of an aqueous solution containing an effective amount of a variant of activated protein C with cytoprotective activity, by way of example APC-2Cys.

In another embodiment of the invention is a method of reducing the incidence of post-surgical adhesions by bathing the internal organs and tissues in an adequate volume of an aqueous solution containing an effective amount of a variant of activated protein C with cytoprotective activity and reduced anticoagulant activity, by way of example APC-2Cys.

The term “reducing the incidence of post-surgical adhesions,” as used herein, refers to preventing, inhibiting, treating, or reducing the incidence of, or reducing the risk of, post-surgical adhesions that may form between internal organs and tissues following general, abdominal, pelvic, abdominopelvic, or cardiac surgery.

The internal organs and tissues to be treated are those that have been exposed by, or have undergone, surgery, as well as adjacent organs and tissues. Preferably, the application of an aqueous solution containing an effective amount of APC, or a variant of APC with cytoprotective properties, is applied subsequent to surgery on, or manipulation of, the internal organs and tissues, and prior to closing the major incision. This surgery on, or manipulation of, the internal organs and tissues, is also referred to herein as the primary surgery. The incision that opens the body cavity providing access to the internal organs and tissues is referred to herein as the major incision. An adequate volume will be determined by the surgeon as that volume necessary to bath the exposed organs and tissues, which will vary depending on the nature of surgery and size of the subject. By way of example, in anticipation of abdominal, pelvic, or chest surgery, a surgeon may determine an adequate volume required to bath the internal organs and tissues of the abdominal, pelvic, or chest cavity. The effective amount is determined based on the subject's body weight and dissolved or diluted in the adequate volume of aqueous solution prior to application. After completion of the primary surgery, the surgeon applies the adequate volume of aqueous solution containing an effective amount of APC or a variant of APC with cytoprotective activity, or a variant of APC with cytoprotective activity and reduced anticoagulant activity, onto the surface of the exposed organs and tissues, as well as generally to the surrounding the abdominal, pelvic, or chest cavity. By way of example, the surgeon may use a syringe or similar device as an applicator to apply an adequate volume of aqueous solution containing APC or variant of APC with cytoprotective properties, onto the organs and tissues in the abdomen, pelvic, or chest region, before closing the abdominal, pelvic, or chest wall with sutures. The solution containing the effective amount of APC, or a variant of APC with cytoprotective activity, or a variant of APC with cytoprotective activity and reduced anticoagulant activity, once applied, may be removed from the subject, but is preferably left in place. Care may be taken by the surgeon to treat all exposed organs, particularly any organs which may have received mild or significant trauma.

Post-surgical adhesions are associated with high mortality and morbidity including intestinal obstruction, pelvic pain, and female infertility. An application of APC as described herein may reduce the incidence of these events. It is also expected that there may be a reduction in the inflammatory events associated with post-surgical adhesions. Non-limiting examples of some indicators of inflammatory events include IL-1, IL-6, TNF-α, TGF-β, and IFN-γ. It was also demonstrated that and increase in tPA was associated with less inflammation and reduced post-surgical adhesions. As described in the examples, these indicators may be measured in any of the appropriate bodily fluid associated with the surgery, particularly in the peritoneal fluid, as possible indicators of abdominal or pelvic abdominopelvic post-surgical adhesions. As demonstrated in the examples it is expected that a reduction in these indicators may represent a reduction in the incidence of post-surgical adhesions. However. It is expected that a reduction in one or more inflammatory indicators, or a relative increase of tPA, in the peritoneal or surgical cavity after surgery relative to typical post-surgical levels, would be indicative of a reduction in the incident of, or risk of formation of, postsurgical adhesions.

### I. Activated Protein C and Activated Protein C-2Cys.

### Protein C (PC) and Activated Protein C (APC)

Wild type human protein C is the inactive zymogen form of a vitamin K-dependent plasma serine protease. Protein C, in vivo, or as secreted by a eukaryotic cell in culture, exists in the form of a disulfide-linked two chain molecule. It is transcribed as a single polypeptide, (see SEQ ID NO:1), which then undergoes post-transitional modification. Modifications include removal of a signal peptide sequence (amino acids 1-42), and removal of a dipeptide sequence (amino acids 198-199), which produces two polypeptides referred to as the light (˜25 kD) (amino acids 43-197) and heavy chains (˜41 kD)(amino acids 200-461). Variations in molecular weight occur due to differences in glycosylation, which is also a post-translational modification. The light chain contains a region of gamma-carboxyglutamic acid, which is required for membrane binding and is dependent on Ca2+. The heavy chain contains the serine protease domain, which also contains a Ca2+ binding site described in detail below. The heavy chain also contains the activation peptide. Activation of protein C to activated protein C takes place in vivo by removal of this activation peptide (amino acids 200-211) by thrombin. A disulfide bond at cysteine 183 and cysteine 319 connects the heavy and light chains. (Plutzky et al., (1986) Proc. Natl. Acad. Sci. (USA) 83, 546-550)

### i) Activation and Anticoagulation Activity

In vivo, protein C circulates as an inactive zymogen. Activation of protein C to activated protein C takes place by proteolytic removal of the activation peptide (amino acids 200-211, see SEQ ID NO:1), from the heavy chain. Protein C is activated on the surface of endothelial cells by a thrombin-thrombomodulin (thrombin-TM) complex, which is also accelerated by the endothelial protein C receptor (EPCR). This is believed to take place by co-locating protein C with the thrombin-TM complex on the endothelial cell surface (Stearns-Kurosawa et al. (1996) Proc Natl Acad Sci. (USA); 93:10212-10216). After activation, activated protein C down-regulates the clotting cascade via a feedback loop mechanism (Stenflo J. (1984) Thromb Hemost. 10:109-121; Esmon C T. (1993) Thromb. Haemost. 70:1-5). Once protein C is activated it may dissociate from EPCR, and form a complex with the vitamin K-dependent protein cofactor, protein S. This complex will shut down the generation of thrombin derived from the cofactor effect of factors Va (fVa) and Villa, which are known to be procoagulant cofactors of the prothrombinase and intrinsic Xase complexes, respectively.

i) Cytoprotective, Anti-Inflammatory Properties, and Anti-Apoptotic Activity

In addition to providing anti-coagulant activity, APC possesses cytoprotective, anti-inflammatory, and anti-apoptotic proprieties. The term “cytoprotective” as used herein is meant to refer collectively to these protective properties or activities, meaning the cytoprotective, anti-inflammatory, and anti-apoptotic proprieties or activities of APC and variants of APC including APC 2Cys. When APC is associated with EPCR, it elicits protective signaling responses in endothelial cells (Taylor et al. (1987) J Clin Invest. 1987; 79:918-925; Taylor et al. (2000) Blood; 95:1680-1686; Joyce et al. (2001) J Biol Chem.; 276:11199-11203; Ruf et al. (2003) J Thromb Haemost. 1:1495-1503; Mosnier et al. (2004) Blood. 104:1740-1744; Finigan et al. (2005) J Biol Chem.; 280:17286-17293). These protective signals may account for the beneficial effects associated with APC when used as an anti-inflammatory agent for treating severe sepsis patients (Bernard et al. (2001) N Eng J. Med.; 344:699-709). The mechanisms of the anti-inflammatory and cytoprotective effects of APC are not well understood, however, it is believed that an APC/EPCR complex cleaves protease-activated receptor-1 (PAR-1) to initiate protective signaling events in endothelial cells (Ruf et al. (2003) J Thromb Haemost. 1:1495-1503; Mosnier et al. (2004) Blood. 104:1740-1744). PAR-1 cleavage by APC may also be required for the inhibition of apoptosis in human brain endothelial cells induced by hypoxia (Cheng et al. (2003) Nature Med.; 9:338-342).

ii) Anticoagulant Activity

The mechanism through which protein C, once activated, functions in the anti-coagulant pathway has been extensively studied and is well understood (Walker et al. (1992) FASEB J; 6:2561-2567). After activation, APC may dissociate, from EPCR and bind to protein S, where it functions as an anticoagulant by degrading factors Va and Villa. Specific recognition of procoagulant factors Va and VIIIa, is determined by the basic residues of an APC exosite (Friedrich et al. (2001) J Biol Chem.; 276:23105-23108; Manithody et al. (2003) 101:4802-4807; Gale et al. (2002) J Biol Chem.; 277:28836-28840). These basic residues are clustered on three exposed surface loops referred to as 37-39 loop, 60-68 loop and 70-80 loop (chymotrypsin numbering system) (Bode et al. (1989) EMBO J.; 8:3467-3475). These basic residues constitute a binding site for TM in the thrombin-TM complex. With the exception of the 60 loop, they are also involved in recognition and subsequent degradation of factors Va and Villa by APC in the anti-coagulant pathway (Friedrich et al. (2001) J Biol Chem.; 276:23105-23108; Manithody et al. (2003) Blood; 101:4802-4807; Gale et al. (2002) J Biol Chem.; 277:28836-28840).

### Protein C-2Cys and Activated Protein C-2Cys

The instant invention utilizes a variant of the wild type protein C as fully described in U.S. Pat. No. 7,785,857, also referred to therein as cross-linked protein C and cross-linked activated protein C. U.S. Pat. No. 7,785,857 is incorporated herein by relevance in its entirety. The instant invention disclosure refers to this variant as protein C-2Cys (PC-2Cys) and activated protein C-2Cys (APC-2Cys) respectively. Protein C-2Cys may be activated by thrombin to form activated protein C-2Cys. Activated protein C-2Cys demonstrates the cytoprotective properties of wild-type APC without the anticoagulant properties.

Protein C-2Cys is produced by engineering a disulfide bond to form a cross-link between two anti-parallel β-sheets of the heavy chain polypeptide of wild-type human protein C. Specifically, amino acid residues at position 264 and also position 279 of wild type protein C (SEQ ID NO:1) are each substituted with a cysteine, to produce a novel polypeptide (SEQ ID NO:2) (Bae et al. (2007) J Biol Chem.; 282: No. 12: 9251-9259). The presence of cysteines in these positions will allow the formation of an intra-chain disulfide bond post-translationally, thereby forming a cross-link between these two amino acids within the anti-parallel β-sheets of residues 261-266 and 278-288 of the protein C heavy chain. Similarly, the presence of cysteines or another cross-linking agent, installed at one or more of these positions, within or near these anti-parallel β-sheets would produce a similar result. Between these anti-parallel β-sheets is the Ca2+ binding 70-80 loop (CHT). While not agreeing to be bound by theory, one hypothesis is that the binding of Ca²⁺ to the 70-80 loop of protein C is associated with a conformational change in the zymogen that is optimal for interaction with thrombin in the presence of TM but inhibitory for interaction in the absence of the cofactor (Yang et al. (2006) Proc Natl Acad Sci. (USA); 103:879-884). By cross-linking these two anti-parallel β-sheets, the 70-80 calcium binding loop becomes stabilized and no longer binds Ca2+. The engineered disulfide bond also stabilizes a Na+ binding site in the high affinity state. These Ca2+ and Na+ binding sites modulate activation of protein C and may be necessary for the amidolytic activity and proteolytic activity demonstrated by APC, as described in detail below. The light chain of Protein C-2Cys is not modified, and as in wild type, remains bound to the heavy chain by a single disulfide bond.

Therefore, by specifically engineering specific regions of the protein C polypeptide, while leaving other regions intact, the anti-coagulant activity of Activated Protein C-2Cys is essentially abolished, while it's anti-inflammatory and cytoprotective signaling properties remain intact.

i) Activation of Protein C-2Cys

Wild type protein C and protein C-2Cys are activated by free thrombin to form activated Protein C and activated protein C-2Cys respectively. Wild type protein C is activated by proteolytic removal of the activation peptide, amino acids 200-211 (SEQ ID NO:1) of the heavy chain. The activation peptide is not altered in protein C-2Cys and activation takes place by removable of the same activation peptide.

ii) Cytoprotective Properties of Activated Protein C-2Cys

As described above, wild type APC possesses protective properties including cytoprotective, anti-inflammatory, and anti-apoptotic activity, collectively referred to herein as cytoprotective properties or cytoprotective activities. These activities are mediated through a receptor designated endothelial protein C receptor (EPCR) present on endothelial cells as well as other cell types. EPCR binding which takes place via the Gla domain (amino acids 43-88 of SEQ ID NO:1) The Gla domain is not altered in Protein C-2Cys, and biological activity of the Gla domain remains unchanged. Therefore, similar to wild type, EPCR binding to endothelial or other cell types via the Gla domain still occurs. Activated Protein C-2Cys participates in cytoprotective, cell signaling, anti-inflammatory and anti-apoptotic activities in the same manner as wild-type APC. These properties are further described and demonstrated in U.S. Pat. No. 7,785,857.

### Method of Making PC-2Cys and APC-2Cys

The method for making PC-2Cys involves methodology that are generally well known and described in detail in numerous laboratory protocols, one of which is Molecular Cloning 3rd edition, (2001) J. F. Sambrook and D. W. Russell, ed., Cold Spring Harbor University Press. Many modifications and variations of the present illustrative DNA sequences and plasmids are possible. For example, the degeneracy of the genetic code allows for the substitution of nucleotides throughout polypeptide coding regions, as well as in the translational stop signal, without alteration of the encoded polypeptide coding sequence. Such substitutable sequences can be deduced from the known amino acid or DNA sequence of human protein C and can be constructed by following conventional synthetic or site-directed mutagenesis procedures. Synthetic methods can be carried out in substantial accordance with the procedures of Itakura et. al., (1977) Science 198:1056 and Crea et. al. (1978) Proc. Natl. Acad. Sci, USA 75:5765. Therefore, the present invention is in no way limited to the DNA sequences and plasmids specifically exemplified.

i) Insertion of Cysteines into Wild Type APC or Derivations of Wild Type APC

A polynucleotide encoding human protein C polypeptide (SEQ ID NO:1) or variants thereof, may be engineered whereby the codons representing one or more of amino acids 261 to 266 and one or more of amino acids 278 to 288 are replaced with codons for cysteine. By way of non-limiting example, and as described below in the examples, nucleotide sequences complementary for a polynucleotide encoding these amino acid sequences may be constructed whereby codons representing amino acid 264 and amino acid 279 are substituted by a cysteine. One of ordinary skill in the art will understand that other codons representing other or additional amino acids within these complementary nucleotide sequences may be replaced with codons for cysteine to provide alternative or additional cysteine substitutions in a similar manner. Examples of primers described below in the examples. The polynucleotide may then be amplified using standard PCR mutagenesis methods as previously described (Rezaie et al. (1992) Journal of Biological Chemistry, vol. 267, pp. 26104-26109) and herein incorporated by reference. The resulting mutant or variant of protein C cDNA may be sub-cloned and inserted into a suitable expressions vector using a number of commercially available restrictions enzymes and expressed in a wide variety of eukaryotic, especially mammalian, host cells. The polynucleotide may be operable linked to a number of suitable control elements to provide an expressible nucleic acid molecule by using standard cloning or molecular biology techniques. See, e.g., Edge (1981) Nature 292:756; Nambair et al. (1984) Science 223:1299; and Jay et al. (1984) J. Biol. Chem. 259:6311. Examples of expression vectors that may be effective for the expression of Protein C-2Cys include, but are not limited to, the PCDNA 3.1, EPITAG, PRCCMV2, PREP, PVAX, PCR2-TOPOTA vectors (Invitrogen, Carlsbad Calif.), PCMV-SCRIPT, PCMV-TAG, PEGSHIPERV (Stratagene, La Jolla Calif.), and PTET-OFF, PTET-ON, PTRE2, PTRE2-LUC, PTK-HYG (Clontech, Palo Alto Calif.). Protein C-2Cys may be expressed using (i) a constitutively active promoter, (e.g., from cytomegalovirus (CMV), Rous sarcoma virus (RSV), SV40 virus, thymidine kinase (TK), or P.beta.actin genes), (ii) an inducible promoter (e.g., the tetracycline-regulated promoter (Gossen et al. (1992) Proc. Natl. Acad. Sci. USA 89:5547-5551; Gossen, M. et al. (1995) Science 268:1766-1769; Rossi et al. (1998) Curr. Opin. Biotechnol. 9:451-456), commercially available in the T-REX plasmid (Invitrogen).

Once constructed, the expression vector encoding Protein C-2Cys may be transfected into host cells using standard gene delivery protocols. Methods for gene delivery are known in the art, and include but are not limited to methods based on naked nucleic acids, calcium phosphate, electroporation, microinjection liposomes, cells, retrovirus including lentiviruses, adenovirus and parvoviruses including adeno-associated virus herpes simplex virus. See, e.g., U.S. Pat. Nos. 7,173,116 6,936,272, 6,818,209, and 7,232,899, which are hereby incorporated by reference. Other gene delivery mechanisms include liposome-derived systems, artificial viral envelopes, and other systems known in the art (See, e.g., Rossi, J. J. (1995) Br. Med. Bull. 51(1):217-225; Boado, R. J. et al. (1998) J. Pharm. Sci. 87; Mosnier et al. (2004) Blood. 104:1740-1744):1308-1315; and Morris, M. C. et al. (1997) Nucleic Acids Res. 25(14):2730-2736).

Techniques for maintaining cells in culture to allow the expression of recombinant polypeptides are well known. By way of example the polynucleotide described above may be expressed in human embryonic kidney cells (HEK-293) using the RSV-PL4 expression system purification vector system as described (Yang et al. (2006) Proc Natl Acad Sci. (USA); 103:879-884) and Yan, U.S. Pat. No. 4,981,952, both of which are hereby incorporated by reference.

Protein C-2Cys may be harvested from the culture media and purified through any combination of protein purification techniques known in the art including various immuno-affinity techniques. An antibody directed to almost any epitope on Protein C-2Cys may be immobilized to a support structure. A physiological solution containing the molecule to be purified is exposed to the antibody whereby the target molecule is bound by the antibody. Methods of releasing polypeptides from antibodies are also well known and may include changes in pH, and elution with various salts, metal ions, EDTA, EGTA, or detergents.

Activated Protein C-2Cys may be produced from protein C-2Cys by incubation with a proteolytic enzyme such as thrombin in a physiological solution. By way of example a solution containing physiological salts and protein C-2Cys may be passed over a column comprising thrombin immobilized to Sepharose. Alternatively, activated protein C-2Cys may be produced directly by expression of a polynucleotide engineered to transcribe an activated protein C-2Cys.

ii) Variants of APC and APC-2Cys

The disclosure of APC and APC-2Cys are meant to be exemplary only. One of ordinary skill in the art will appreciate that variants of APC or APC-2Cys will be effective in reducing the incidence of post-surgical adhesions provided they retain cytoprotective properties or activities such as antiinflammatory and anti-apoptotic activities. Variants of APC may be modified by the insertion of cysteines in the 70-80 calcium binding loop, by way of example at positions 264 and 279 to eliminate anticoagulant activity. Variants of APC may also be produced by the insertion, deletion or substitution, of one or more amino acids at other sites in APC provided the resulting polypeptide retains cytoprotective activity. Variants of APC may also be produced by applying similar modifications to APC-2Cys, or other existing variants of APC. It is expected that these modified derivative polypeptides would possess the same or similar anti-adhesive activity as has been demonstrated for APC and PC-2Cys, provided the resulting polypeptide retains cytoprotective activity.

Examples of variants or derivatives of APC that may be useful in reducing the incidence of post-surgical adhesions are any variants or mutants of APC which possess cytoprotective, cell signaling, anti-inflammatory or anti-apoptotic activities including those described by Gerlitz, et al., U.S. Pat. No. 5,453,373, and Foster, et al., U.S. Pat. No. 5,516,650, the entire teachings of which are hereby included by reference.

Other examples of variants or mutants or derivatives of APC, or APC-2Cys include APC or APC-2Cys with conservative amino acids substitutions that retain cytoprotective properties such as antiinflammatory and anti-apoptotic activities, regardless of the presence or absence of anticoagulant activity. These variants, mutants, or derivatives of APC, or APC-2Cys are expected to be effective in preventing post-surgical adhesions when applied according to the methods described herein.

### II. Formulations and Administration of Activated Protein C-2Cys (APC-2Cys)

### A. Pharmaceutical Dosage Form

Pharmaceutically acceptable formulations are well known for use as injectable solutions of APC. It is expected that most known injectable physiological solutions or formulations will be acceptable for the topical peritoneal application described herein. However, the amounts of APC per milliliter of solution may vary widely. By way of example, an effective amount of APC which may be obtained commercially as a sterile solution and diluted into a larger volume, or adequate volume, of sterile aqueous solution to be applied as described herein.

APC is a hydrophilic polypeptide and may be administered in a sterile aqueous solution, preferably in a physiological solution. A physiological solution may be comprised of isotonic balanced salts with a pH of about 7.0 to about 7.5. A preferred physiological solution may comprise isotonic saline and a pH of 7.5.

The aqueous solution may further contain various salts or buffers that are well known in the art. Injectable preparations, for example, sterile aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent. Non-limiting examples of acceptable vehicles and solvents that may be employed include, Ringer's solution, or isotonic sodium chloride solution.

It is preferable to maintain the pH in a physiological range, from about 6.0 to about 6.5, or about 6.5 to about 7.0, or about 7.0 to about 7.5, or about 7.5, to about 8.0, or most preferably about 7.0 to about 7.5. To maintain effective pH control, the activated protein C solution should contain a pharmaceutically acceptable buffer.

Similarly, it is preferable to maintain the ionic strength in a physiological range. The ionic strength is generally determined by the salt concentration of the solution. Pharmaceutically acceptable salts typically used to generate ionic strength include, but are not limited to, potassium chloride (KCl) and sodium chloride (NaCl). The preferred salt is maintained in a physiological range; for example, sodium chloride may be used at a concentration of 0.9 percent by weight or 100 to 150 mM.

Formulations developed for activated protein C are also known in the art and including those described in U.S. Pat. Nos. 6,630,137, 6,159,468, and 6,359,270 which are hereby incorporated by reference. Activated protein C may be formulated to prepare a pharmaceutical composition comprising as the active agent, protein C or activated protein C, and a pharmaceutically acceptable solid or carrier. For example, a desired formulation would be one comprising a bulking agent such as sucrose, a salt such as sodium chloride, a buffer such as sodium citrate and activated protein C. Formulations may be lyophilized for storage, and hydrated before use. Examples of stable lyophilized formulations include 5.0 mg/ml activated protein C, 30 mg/ml sucrose, 38 mg/ml NaCl and 7.56 mg/vial citrate, pH 6.0; and, 20 mg/vial activated protein C, 120 mg/ml sucrose, 152 mg/vial NaCl, 30.2 mg/vial citrate, pH 6.0.

Alternatively, APC formulated into pharmaceutical compositions are administered by a number of different means that will deliver a therapeutically effective dose. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

### B. Administration of Therapeutically Effective Amounts

The Inventors have previously demonstrated effective amounts of wild type APC used to reduce the incident of post-surgical adhesions (see U.S. application Ser. No. 13/854,117, incorporated herein by refinance in its entirety). It was expected that effective amounts of wild type APC and APC-2Cys would be the same or similar, and this has been demonstrated in the examples below. It is also expected that effective amounts of other variants of APC with cytoprotective properties will be the same or similar to those observed for wild type APC or APC-2Cys, since the functional region or activity of the polypeptide responsible for the anti-adhesive activity is not altered. A therapeutically effective amount, also referred to herein as an effective amount of APC, or a variant of APC with intact cytoprotective properties, by way of example APC 2Cys, may be determined by a skilled practitioner, typically a medical doctor, and typically based on based on a subject's weight. The skilled practitioner may also monitor post-surgical events in the same or similar subjects and adjust the effective amount in subsequent surgeries. Examples of post-surgical events that may be monitored include levels of the inflammatory indicators as described herein, namely II-1, II-6, TNFα, TGFβ and IFN-γ in addition to tPA. Inflammatory indicators are preferably monitored in the appropriate surgical body cavity fluid. By way of non-limiting example, appropriate surgical body cavity fluids include: peritoneal fluid for abdominal, pelvic, or abdominopelvic surgery; pericardial fluid for cardiac surgery; and pleural fluid for thoracic surgery. Post-surgical events that may be monitored also include the incidence of symptoms of post-surgical adhesions: by way of example abdominal, pelvic, or thoracic pain; intestinal obstructions; female infertility; and adhesion of the heart to surrounding tissues. The skilled practitioner may increase the effective amount of APC or a variant of APC with intact cytoprotective properties, by way of example APC-2Cys used in subsequence surgeries, in the same or different subjects, if a further reduction in the incidence of post-surgical adhesions is required.

A therapeutically effective amount or an effective amount may be administered in an appropriate volume of aqueous solution and used to bath the internal organs and tissues as well as the appropriate body cavity, by way of example the peritoneal cavity. An effective amount may be calculated based on the subject's body weight. By way of example, an effective amount may be between about 0.01 and about 1000 micrograms per kilogram of the subject being treated. A preferred effective amount is between about 1 and about 500 micrograms per kilogram of the subject being treated. A more preferred effective amount is between about 10 and about 200 micrograms per kilogram of the subject being treated. Another more preferred effective amount is between about 25 and about 200 micrograms per kilogram of the subject being treated. Another more preferred effective amount is between about 25 and about 100 micrograms per kilogram of the subject being treated. Yet another more preferred effective amount is between about 25 and about 50 micrograms per kilogram of the subject being treated. A most preferred effective amount is about 50 micrograms per kilogram of the subject being treated.

An appropriate volume of aqueous solution is that which would be required to bath the organs and tissues of the body cavity of a particular subject. The appropriate volume of aqueous solution will vary according to the size of the subject, the size of the abdominal, pelvic, or chest incision, and the type of surgery. Surgeries that expose more internal organs will require larger volumes of solution. If desired the surgeon may apply a sterile balanced salt solution to the subject in order to estimate the required or appropriate volume. The appropriate volume of aqueous solution may be determined or estimated and used to dissolve or dilute an effective amount of the variant of APC with cytoprotective activity, or the variant of APC with cytoprotective activity and reduced anticoagulant activity, by way of example APC-2Cys. The appropriate volume of aqueous solution containing the variant of APC may then be applied to the internal organs and generally to the appropriate body cavity of the subject.

It is expected that the appropriate volume of aqueous solution containing the variant of APC with cytoprotective activity, or the variant of APC with cytoprotective activity and reduced anticoagulant activity, by way of example APC-2Cys, would be administered near the completion of any general, abdominal, pelvic, or chest surgery prior to closing the major incision. An aqueous solution containing an effective amount of the variant of APC may be applied to the exposed organs and lining of the particular body cavity after completion of the primary surgery but prior to closing the major incision. Particular attention may be directed to exposed organs and those suspected of receiving mild or significant trauma during surgery. It is anticipated that any or all internal organs may receive treatment, including but not limited to the cecum and rectum, spleen, liver, lungs, reproductive organs, and heart. It is anticipated that the type of surgery may make special attention to certain organs appropriate. By way of non-limiting examples, abdominal surgery may suggest that special attention be paid to organs of the peritoneal cavity. Gastro-intestinal surgery may suggest that special attention be paid to organs of the gastro-intestinal track, including the cecum and rectum. Gynecological surgery may suggest that special attention be paid to the uterus, fallopian tubes, or ovaries. Cardiac surgery may suggest that special attention be paid to the heart and surrounding tissues, including the pericardium. In general, surgeries that involve the manipulation of certain organs and tissues suggest that special attention should be applied to those same organs and tissues, as well as adjacent organs and tissues in order to ensure that they are adequately treated with the aqueous solution containing a variant of APC with cytoprotective activity, or a variant of APC with cytoprotective activity and reduced anticoagulant activity, by way of example APC-2Cys. Regardless of the type of the surgery, it is recommended that a solution containing the variant of APC, be applied to any site where post-surgical adhesion may develop, and since it is possible that almost any site has the potential to develop post-surgical adhesions, it is preferred that the treatment be applied as extensively as possible.

### III. Subjects and Types of Surgeries

Subjects, as used herein, are meant to include human and non-human mammals. It is expected that human subjects will benefit greatly from the invention. It is also expected that non-human mammalian subjects, including domestic animals, experimental animals, or livestock, will also benefit greatly from the invention. Since all subjects undergoing a general, abdominal, pelvic, abdominopelvic, thoracic, or cardiac surgery may develop post-surgical adhesions, it is anticipated that any subjects undergoing general, abdominal, pelvic, abdominopelvic, thoracic or cardiac surgery may benefit form topical application of APC, or a variant of APC with cytoprotective activity, or a variant of APC with cytoprotective activity and reduced anticoagulant activity, by way of example APC-2Cys, to the internal organs and tissues. Therefore all subjects undergoing general, abdominal, pelvic, or cardiac surgery are candidates for treatment. It is believed that this method of treating post-surgical adhesions may be applied generally to all surgeries where post-surgical adhesions occur. It is also expected to benefit applications where Seprafilm may be applied.

It is known that post-surgical adhesions occur following cardiac surgery. Seprafilm has been shown to have a beneficial effect following cardiac surgery in a canine abrasion/desiccation pericardial adhesion model (Seeger et al., (1997) Journal of Surgical Research, 68:63-66). In light of the Inventor's demonstration that the beneficial anti-adhesive effect of APC is significantly greater than HA/CMC in the organs of the abdominal cavity, it is expect that the anti-adhesive effect of APC in preventing post-surgical cardiac adhesion band formation will also be significantly greater than HA/CMC.

The discussion of the references herein is intended merely to summarize the assertions made by the authors and no admission is made that any reference constitutes prior art. Applicants reserve the right to challenge the accuracy and pertinence of the cited references.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

The anti-adhesive effects of wild-type recombinant APC, 3 APC variants including APC-2Cys, and the FDA approved drug sodium hyaluronate/carboxymethylcellulose (HA/CMC, aka Seprafilm), was compared by administering, intraperitoneally, into 60 male C57BL/6 mice. After 7 days, the pathological adhesion grades were scored using the Nair and Leach scaling systems. The concentrations of the proinflammatory cytokines II-1, II-6, TNFα, TGFβ and IFN-γ were evaluated. Inflammation scores of mice in all groups were determined.

### Methods and Materials

Sixty male C57BL/6 mice, weighing from 25 g to 35 g, were randomly divided into 6 groups: group 1, control animals with surgical abrasion and no treatment (n=10); group 2, surgical abrasion plus HA/CMC (Seprafilm, Genzyme Biosurgery Corporation, Cambridge, Mass.) as a gold standard (n=10); group 3, surgical abrasion plus 50 microgram/kg of activated protein C (wild type APC) intraperitoneally (i.p) (n=10); group 4, surgical abrasion plus 50 μg/kg of APC-2Cys administered intraperitoneally (i.p.) (n=10); group 5, surgical abrasion plus 50 μg/kg of APC-E170A administered intraperitoneally (i.p.) (n=10) and group 6, surgical abrasion plus 50 μg/kg of APC-S195A administered intraperitoneally (i.p.) (n=10). The surgical treatment was performed under a general anesthesia inhalant containing ethoxyethane (ether) and intraperitoneal ketamine hydrochloride (ketamine hydrochloride 50-mg/vial; DeltaSelect; DeltaSelect GmbH, Otto-Hahn-Straβe, Dreiech, Germany) 3 mg/kg. All animal cares and procedures were performed according to the guidelines approved by the ethical committee of St. Louis University Medical School.

### Surgical Technique and Administration of APC and APC Variants

The Inventors used the adhesion induction model previously described by Hemadeh et al (Hemadeh et al., (1993) Surgery, 114:907-10), which resulted in a 100% occurrence of surgical adhesions in the controls after 7 days. After anesthesia, the skin of abdomen was shaved and cleaned with povidone iodine solution. A vertical midline incision of 2.5 cm was made and abdomen was opened. To induce adhesion band formation, the cecum was exposed, and rubbed gently with 2 dry gauze pads at the ventral and dorsal surfaces until it lost its shine and hemorrhagic points became visible. The cecum was then returned to its anatomic position in abdominal cavity. Before closing the abdominal cavity, the Inventors administered intraperitoneally APC or Seprafilm.

APC and APC variants were administered as follows. After induction of adhesion bands at the cecum, as described above, a single dose of 50 microgram/kg of APC (Drotrecogin Alfa (Activated)), APC-2Cys, APC-E170A, or APC-S195A, in 1 ml of saline solution was administered locally to exposed organs in the peritoneal cavity. Particular efferent was made to insure the site of surgery receiving a portion of this topical administration. After application, the abdomen cavity was then closed. Seprafilm was also administered as physical barrier between abdominal wall and exposed organs and also between organs.

After 7 days, mice euthanized and their abdominal cavities were opened by two surgeons who were blinded with regard to previous treatment received by each animal. The mice were scored according to the Nair and Leach scales set forth in Table 1.

TABLE 1
Adhesion and Inflammation Scores Classification.
The Table is adopted from Lalountas (Lalountas et al. (2010)
The American Journal of Surgery 200, 118-123).

| Grade | Nair et al* | Leach et al* | Inflammation |
|---|---|---|---|
| 0 | Complete absence of | No adhesion | Nill |
| | adhesions | | |
| 1 | Single band of | Filmy thickness, | Giant cells, |
| | adhesions, between | avascular | occasional scattered |
| | viscera, or from 1 | | lymphocytes |
| | viscus to | | and plasma cells |
| | abdominal wall | | |
| 2 | Two bands: | Limited vascularity, | Giant cells with |
| | between viscera | moderate thickness | increased numbers |
| | or from viscera | | of admixed |
| | to abdominal | | lymphocytes, |
| | wall | | plasma cells, |
| | | | eosinophils and |
| | | | neutrophils |
| 3 | More than 2 bands: | Well vascularized, | Many admixed |
| | between viscera, or | dense thickness | inflammatory cells, |
| | viscera to abdominal | | micro abscesses |
| | wall, or whole of | | present |
| | intestines forming | | |
| | a mass without | | |
| | being adherent to | | |
| | abdominal wall | | |
| 4 | Viscera directly | — | — |
| | adherent to abdominal | | |
| | wall, irrespective of | | |
| | number and extent | | |
| | of adhesive bands | | |

*Refs. (Nair et al., (1974) Arch Surg; 108: 849-53; Leach et al., (1998) Fertil Steril 69: 415-8)

### Statistical Analysis

All variables were expressed as mean±SD and ranks. Differences between the adhesion and inflammation scores of study groups were evaluated by Kruskal-Wallis variance analysis and when significant (P<0.05), the difference between specific mean ranks was determined using the multiple comparison procedure based on ranks. The differences between ELISA samples were also evaluated by one-way Anova test. The level of significance was set to 5%.

### Inflammation

Inflammatory Cells in Peritoneal Cavity Fluids

Because the pathologic mechanism of adhesion band formation is known to involve inflammation, peritoneal inflammatory cells were monitored by flow cytometry. Multiple specific antibodies (see Table 2) were used to detect inflammatory cells in the peritoneal cavity fluids in order to monitor the relationship between these inflammatory cells and amount of adhesions in each group.

TABLE 2
Antibodies used to monitor inflammation

| Specific Antibody (Flow Cytometry) | Specific Cell Detection |
|---|---|
| Alexa Fluor ® 700 anti-mouse | All White Blood Cells |
| CD45Antibody | |
| Pacific Blue ™ anti-mouse CD3 Antibody | T cells |
| PE/Cy7 anti-mouse CD19 Antibody | B cells |
| PerCP/CyS.S anti-mouse/human CDIIb | Myeloid cells, Macrophages |
| | Antibody |
| FITC anti-mouse Ly-6C Antibody | Neutrophils |
| PE anti-mouse CDIIc Antibody | Dentritic cells |
| APC anti-mouse TER-119/Erythroid | Red Blood Cells |
| Cells Antibody | |
| Purified anti-mouse CD16/32 Antibody | For Blocking the samples |

Enzyme-Linked Immunosorbent Assays (ELISA)

Commercially available ELISA kits were used to measure the peritoneal fluid concentrations of cytokines, IL-6, TNF-α, IL-1β, IFN-, and TGF-β1 (eBioScience, San Diego, Calif.) according to the manufactures' instructions. Thrombin-Antithrombin (TAT) complex which has been measured in plasma. The ELISA kit for measuring the concentration of tissue plasminogen activator (tPA) was obtained from Innovative Research Inc. (Novi, Mich.). Thrombin-Antithrombin complex mouse elisa kit was purchased from Abcam. The animals (N=5 in each bar) were used for ELISA measurement, were not used for adhesion grade evaluation.

Real-time PCR analysis-Quantitative real-time PCR was performed using Premix Ex Taq (TAKARA Bio) on an Applied Biosystems 7300 real-time PCR system (Applied Biosystems) or a StepOnePlus system (Applied Biosystems), as described previously with some modification. To determine mRNA level of ICAM-1, VCAM-1, MCP-1, MMP-2 and TIMP-2 expression levels (FIG. 4), gene-specific primers and probes were used. Expression of each mRNA was normalized for glyceraldehyde-3-phosphate dehydrogenase using TaqMan Rodent glyceraldehyde-3-phosphate dehydrogenase control reagents (Applied Biosystems).

Histological evaluation. Membranes and the surrounding peritoneal tissues were separated from other tissues, fixed in 10% formalin and immersed in paraffin. Several paraffin sections were made by microtome. These sections were 16 stained by Haematoxylin-Eosin (HE) and the degree of inflammation was evaluated in a double blind-study by two assessors using a semi-quantitative scoring system as described previously (see Dinarvand, et al. (2012) J Surg Res 172:e1-9 and Hooker et al., (1999) Surgery 125:211-216).

### Production of APC 2-Cys: Construction and Expression of Recombinant Proteins

Then production of APC-2Cys was performed as described in U.S. Pat. No. 7,785,857, referred to therein as cross-linked APC. U.S. Pat. No. 7,785,857 is incorporated herein by reference in its entirety, The amino acid sequence for Protein C has previously been described (Beckmann et al. (1985) Nucleic Acids Res. vol. 13 pp. 5233-5247; Plutzky et al. (1986) PNAS Vol. 83, pp 546-550), and is represented by SEQ ID NO:1 (Accession number NP_{—}000303, NBCI). Elements of the inventors' methodology not described herein are generally well known and detailed in numerous laboratory protocols, including Molecular Cloning 2nd edition, (1989) Sambrook, J., Fritsch, E. F., and Maniatis, J., Cold Spring Harbor., and Current Protocols in Molecular Biology, volumes 1-3, John Wiley & Sons, Inc. herein incorporated by reference.

Wild-type protein C (SEQ ID NO:1) and the Protein C-2Cys, (SEQ ID NO:2) were expressed in human embryonic kidney cells (HEK-293) by using the RSV-PL4 expression system purification vector system as described (Yang et al. (2006) Proc Natl Acad Sci. (USA); 103:879-884) and herein incorporated by reference. Two complementary sense 5′-AAG AAG CTC CTT GTC TGC CTT GGA GAG TAT GAC-3′ (SEQ ID NO:3) and antisense 5′-GTC ATA CTC TCC AAG GCA GAO AAG GAG CTT CTT-3′ (SEQ ID NO:4) oligonucleotide PCR primers representing the three base codons for the amino acid residues 62-72 (chymotrypsin numbering) were synthesized in which the codon for Arg-67 (residue 264 of SEQ ID NO:1) was replaced with the codon for cysteine in both primers (underlined). Moreover, two additional oligonucleotides 5′-GAG AAG TGG GAG CTG TGC CTG GAO ATC AAG GAG-3′ (SEQ ID NO:5) (sense) and 5′-CTC CTT GAT GTC CAG GCA CAG CTC CCA CTT CTC-3′ (SEQ ID NO:6) (anti-sense) representing amino acid residues 77-87 were synthesized in which the codon for the residue Asp-82 (residue 279 of SEQ ID NO:1) was replaced with the codon representing cysteine in both primers (underlined) The protein C cDNA (SEQ ID NO:7) (Accession number NM_{—}000312) was amplified in two rounds to incorporate the desired mutations into the protein C sequence using standard PCR mutagenesis methods as previously described (Rezaie et al. (1992). Journal of Biological Chemistry, vol. 267, pp. 26104-26109) and herein incorporated by reference. The resulting mutant protein C cDNA (SEQ ID NO:8) was sub-cloned into HindIII and XbaI restriction enzyme cloning sites of the commercially available expression vector pRc/RSV (Invitrogen, San Diego, Calif.) using standard cloning methods. This vector contains a G418 resistant gene for selection in mammalian cells using the aminoglycoside antibiotics Gentamycin (Calbiochem, San Diego, Calif.). The accuracy of the mutations in the expression vector containing the mutant cDNA was confirmed by DNA sequencing and then introduced to human embryonic kidney (HEK) 293 cells for expression. A high expressing stable G418 resistant clone was identified by a Sandwich ELISA using an anti-protein C polyclonal antibody and the HPC4 monoclonal antibody, and expanded for production as described (Ref. Journal of Biological Chemistry, A. R. Rezaie and C. T. Esmon, vol. 267, pp. 26104-26109, 1992). The mutant protein APC-2Cys (SEQ ID NO:2) was isolated from 20-L cell culture supernatants by a combination of immunoaffinity and ion exchange chromatography using the HPC4 monoclonal antibody and a Mono Q ion exchange column (Amersham Pharmacia). The preparation of wild-type protein C and protein C-2Cys as well as APC-E170A and APC-S195A are described in Refs. Bae J S, Yang L, Manithody C, and Rezaie A R. (2007), J. Biol. Chem. 282:9251-9259 and Bae J S, Yang L, Manithody C, and Rezaie A R (2007) J. Biol. Chem. 2007, 282(35):25493-25500, incorporated herein by reference in their entirety.

### Example 1

### Adhesion Bands

The grades of post-surgical intraperitoneal adhesion bands (frequency and distribution) in experimental mice were measured after 7 days according to two methods described by Nair (Nair et al., (1974) Arch Surg; 108:849-53) and Leach (Leach et al., (1998) Fertil Steril 69:415-8) (FIG. 1). There were no mortalities due to anesthetic induction or surgical procedures. No serious complications, such as bleeding related to the dosage of APC required to reduce adhesion formation, was detected. The grades of adhesion bands (frequency and distribution) were measured and the Nair et al scaling method was applied as presented in Table 3.

The results obtained from the APC and APC-2Cys-treated groups were virtually identical with regard to adhesion formation and inflammatory response. Both APC and APC-2Cys were administered at an identical dose of 50 ug/kg body weight and effectively inhibited post-surgical adhesion band formation in the experimental animals. By contrast, nearly half of the adhesion bands remained in the Seprafilm treated group. As demonstrated in FIG. 2, both wild-type APC and APC-2Cys equally and effectively inhibited the formation of post-surgical adhesion band formation as determined by both Nair and Leach scoring system, and were significantly better than Seprafilm (HA/CMC). Intraperitoneal administration of wild-type APC and APC-2Cys resulted in a much higher reduction of adhesion band formation (P<0.05) relative to the HA/CMC treatment group (see FIG. 2 and Table 3). By contrast, the APC-E170A which does not have cytoprotective activity, did not exhibit superior anti-adhesion properties when compared to Seprafilm. In addition, the APC-S195A variant of APC, which possesses neither anticoagulant nor cytoprotective activity, exhibited no anti-adhesion properties (FIG. 2)

TABLE 3
Mean adhesion scores in mice as determined
by Nair et., and Leach et al..
Nair-mean Leach-mean Adhesion band Scores

| | Nair | Leach |
|---|---|---|
| Control | 2.8 | 2.1 |
| Seprafilm | 1.4 | 1.1 |
| APC-WT | 0.4 | 0.3 |
| APC-2cys | 0.3 | 0.2 |
| APC-E17A | 1.5 | 1.1 |
| APC-S195A | 2.8 | 2.2 |

### Example 2

### Inflammation

The analysis of peritoneal cavity fluids by flow cytometry using antibodies directed at specific cellular markers indicated that the total number of white blood cells decreased in the wild-type APC and APC-2Cys-treated groups (see Table 4). The results indicate that both APC and APC-2Cys, but not Seprafilm inhibits the migration of inflammatory cells, neutrophils, to peritoneal cavity of experimental animals.

This analysis indicates that the percentage of neutrophils in both cases were half that indicated in untreated saline and Seprafilm treated groups. Interestingly, the percentage of dendritic cells was increased in both APC and APC-2Cys groups, possibly suggesting a protective role for these cells in this inflammatory model. Similar to untreated control, neither APC-E170A nor APC-S195A inhibited the migration of neutrophils to peritoneal cavity as their number remained elevated in both groups. These results clearly show the inhibitory effect of APC and APC-2Cys on migration of inflammatory cells to the peritoneal cavity in response to injury. Another interesting observation is that the number of red blood cells (RBC) detected in the peritoneal cavity fluid did not increase in any of APC-treated groups, suggesting that treatment with APC is not associated with increased incidence of bleeding.

TABLE 4
Percentages of cells detected in peritoneal fluid using Flow Cytometry

| Groups | Amount of Cells (Peritoneal samples) |
|---|---|
| Negative Control | WBC: 91%, RBC: 7.6%, T cell: 14%, |
| | B cell: 18%, Dentritic cell: 4%, |
| | Neutrophils: 41.6%, Macrophages: 27% |
| Positive Control (Seprafilm) | WBC: 91%, RBC: 6.7%, T cell: 11%, |
| | B cell: 15%, Dentritic cell: 4%, |
| | Neutrophils: 44%, Macrophages: 28% |
| APC-Wild Type | WBC: 82%, RBC: 3.7%, T cell: 19.4%, |
| | B cell: 28%, Dentritic cell: 7.15%, |
| | Neutrophils: 20.5%, Macrophages: 21% |
| APC-2cyc | WBC: 81%, RBC: 4.8%, T cell: 15.4%, |
| | B cell: 33%, Dentritic cell: 6.30%, |
| | Neutrophils: 19%, Macrophages: 22% |
| APC-E170A | WBC: 94.6%, RBC: 4.6%, T cell: 17%, |
| | B cell: 17%, Dentritic cell: 5.5%, |
| | Neutrophils: 41%, Macrophages: 13.8% |
| APC-S195A | WBC: 93.5%, RBC: 3%, T cell: 13%, |
| | B cell: 18.6%, Dentritic cell: 4%, |
| | Neutrophils: 39%, Macrophages: 31.8% |

### Example 3

### Analysis of Proinflammatory Cytokines

The results presented in FIG. 3 demonstrates that both wild-type APC and APC-2Cys equally and effectively inhibit the production of IL-6 in the peritoneal fluid cavity of experimental animals at all three time points of 6 h, 24 h and one week after surgery. By contrast, Seprafilm has no effect on inhibiting this proinflammatory cytokine during the same time period. Consistent with inflammation scores, APC-E170A was less effective and APC-S195A had no inhibitory activity on IL-6 production. Similarly, the results presented in FIG. 3 demonstrates that both wild-type APC and APC-2Cys equally and effectively inhibit the production of TNF-α in the peritoneal fluid cavity of experimental animals at all three time points of 6 h, 24 h and one week after surgery. However, Seprafilm has no effect on inhibiting this proinflammatory cytokine during the same time period. Consistent with results of other figures, APC-E170A was less effective and APC-S195A had no inhibitory effect on TNF-α production.

Peritoneal fluid concentrations of different cytokines were determined by ELISA at four time points of 0 h, 6 h, 24 h and one week post-surgery (FIG. 3). At the 6 h time point, the IL-1 level was elevated greater than 6-fold in the control group and treatment with either Seprafilm or APC did not result in any statistically significant effect in the cytokine level (FIG. 3A). The IL-1 level gradually declined and reached near the baseline level one week post-surgery (FIG. 3A). By contrast, the concentration of IL-6 was elevated in both the control and Seprafilm-treated groups, however, the IL-6 level was markedly inhibited in the APC-WT and APC-2Cys treated groups 6 h post-surgery (FIG. 3B). Interestingly, there was also a modest decrease in the IL-6 level in the APC-E170A treated group (FIG. 3B). While the IL-6 level was decreased nearly to a baseline level in the APC-WT and APC-2Cys treated group, the cytokine level remained modestly elevated with all other groups one week post-surgery. The IL-6 level was not affected by Seprafilm and remained elevated for the duration of the experiment (FIG. 3B). Similarly, APC-WT and APC-2Cys effectively inhibited TNF-α, thus decreasing its secretion to a near baseline level at all three time points examined (6 h, 24 h and one week) (FIG. 3C). By contrast, the TNF-α level remained high in all others groups including the Seprafilm group for one week post-surgery (FIG. 3C). While Seprafilm had no effect on the expression of TGF-β, APC-WT and APC-2Cys dramatically inhibited its secretion at both 6 h and 24 h time points (FIG. 3D). APC-E170A also exhibited a modest TGF-β inhibitory effect (6 h and 24 h), however, APC-S195A had no effect in preventing surgery-mediated peritoneal TGF-β expression (FIG. 3D). The APC variants exhibited a similar activity profile toward IFN-γ, however, Seprafilm had no significant effect on the IFN-γ expression level, and thus its level remained high for the duration of the experiment (FIG. 3E). Interestingly, the concentration of tPA in peritoneal fluids was markedly elevated in the APC-WT and APC-2Cys treated groups 6 h post-surgery (FIG. 3F). Similar to results presented above, APC-E170A also modestly increased tPA secretion, but Seprafilm had no effect on the tPA level for the duration of experiments (FIG. 3F). None of the APC variants had a significant modulatory effect on the coagulation cascade as determined by the levels of thrombin-antithrombin (TAT) complex in plasma samples measured at different time points (FIG. 3G). Taken together, the results indicate that the effectiveness of APC-WT and APC-2Cys in inhibiting post-surgical proinflammatory responses and thereby preventing adhesion band formation is markedly superior to that observed with Seprafilm. The results further indicate that the cytoprotective or signaling activity of APC is primarily responsible for its antiinflammatory properties.

### Example 4

Expression of cell adhesion molecules. Finally, the results in FIG. 4 demonstrate that APC-2Cys effectively inhibits the mRNA expression of cell adhesion molecules vascular cell adhesion molecule 1 (VCAM-1), intracellular cell adhesion molecule 1 (ICAM-1), and proinflammatory MCP-1 and matrix metalloproteinase 2 (MMP-2). By contrast, Seprafilm had minimal effect on the mRNA expression of these proinflammatory molecules. Also, APC-2Cys promoted the mRNA expression of the tissue inhibitor of metalloproteinase 2 (TIMP-2), but Seprafilm had no effect on the mRNA expression of TIMP-2 (FIG. 4, panel E). The cytoprotective signaling activity of wild-type APC on the mRNA expression of molecules presented on FIG. 4 was identical to the activity of APC-2Cys, but neither APC-E170A nor APC-S195A exhibited cytoprotective activity in this assay (data not shown).

### Example 5

Histological evaluation. The histology of peritoneal band tissues in two magnifications for different groups: saline treated control (A), Seprafilm (B), APC-WT (C), APC-2Cys (D), APCE170A (E) and APC-S195A (F) are presented in FIG. 5. The inflammation scores, evaluated by two assessors in a double blind-study based on the histological data, are presented in FIGS. 5G and H. In the saline treated control group, many inflammatory cells were recruited to peritoneal tissues and some micro abscesses could also be observed (FIG. 5A). The same cell types, but to a lesser extent, could also be detected in the photomicrographs of the Seprafilm-treated animals (FIG. 5B). In contrast to both the control and Seprafilm groups, the number of inflammatory cells were markedly decreased and essentially no micro abscesses could be observed in the APC-WT and APC-2Cys treated groups (FIGS. 5C, and D). There were only a few scattered leukocytes in the APC-treated groups. The inflammation scores determined by two assessors in a double blind study on a scale of 0-3 (see Dinarvand, et al. (2012) J Surg Res 172:e1-9 and Hooker et al., (1999) Surgery 125:211-216) are presented in FIG. 5G. Statistically significant differences were observed between mice receiving either Seprafilm or APC-WT, APC-2Cys and APC-E170A derivatives and those receiving saline or APC-S195A (p<0.05). In comparison with the Seprafilm and APC-E170A, a significant decrease of the inflammation score (p<0.05) was observed for the APC-WT and APC-2Cys groups (FIG. 5G). The significantly lower inflammation scores of the two APC derivatives are consistent with their dramatic inhibitory effect on the expression levels of proinflammatory cytokines as demonstrated above in the figures above.

### CONCLUSION

These results demonstrate that both wild-type APC and the APC-2Cys variant lacking anticoagulant activity exhibited the same high efficacy in preventing post-operative adhesion band formation. In addition, this level of efficacy was much higher than that observed for the FDA approved drug, Seprafilm. Both APC and APC-2Cys hold the potential to be therapeutically effective in reducing the incidence of post-surgical adhesion band formation. It is also noted that mice treated with APC-E170A (lacking signaling activity) and APC-S195A (lacking both signal activity and anticoagulative activity) were not effective in inhibiting post-surgical adhesions. It can be concluded that the anti-adhesive activity of APC or APC-2Cys is mediated via its cytoprotective and antiinflammatory properties and that the anticoagulant activity of APC is not required for anti-adhesive activity. It therefore follows that any variant of APC or APC-2Cys in possession of cytoprotective and antiinflammatory activity will be effective in inhibiting post-surgical adhesions, regardless of the presence or absence of other APC properties, such as anticoagulant activity. It is believed any variant of APC or APC-2Cys with cytoprotective properties can be used as a therapeutic anti-adhesive drug for the reduction of post-surgical adhesion band formation as it occurs in the internal organs after general, abdominal, pelvic, or cardiac surgeries. It is also believed that a variant of APC with cytoprotective activity and reduced anticoagulant activity such as APC-2Cys would be especially beneficial due to its anti-adhesive effect and reduced risk for bleeding. In addition, APC and APC 2Cys dramatically inhibited the secretion of II-6, TNFα, TGFβ and IFN- in peritoneal tissues, which was associated with reduced post-surgical band formation. It is anticipated that a reduction of these as well as other inflammatory indicators may be used as markers of reduced post-surgical band formation.

All publications and patents cited in this specification are hereby incorporated by reference in their entirety. The discussion of the references herein is intended merely to summarize the assertions made by the authors and no admission is made that any reference constitutes prior art. Applicants reserve the right to challenge the accuracy and pertinence of the cited references.

What is claimed is:

1. A method of reducing the incidence of post-surgical adhesions in a subject undergoing surgery, the method comprising,
a) completing a primary surgery,
b) topically administering an effective amount of a variant of activated protein C comprising cytoprotective activity, to the organs and tissues exposed by the primary surgery or subjected to surgical manipulation by the primary surgery, and
c) closing a major incision associated with the primary surgery.

2. The method of claim 1, wherein the variant of activated protein C comprising cytoprotective activity further comprises reduced anticoagulant activity.

3. The method of claim 2, wherein the variant of activated protein C comprising cytoprotective activity consists of the polypeptide set forth in SEQ ID NO: 2, as secreted from a eukaryotic cell and further activated with thrombin.

4. The method of claim 1, wherein the effective amount is about 25 micrograms per kilogram to about 100 micrograms per kilogram of the subject being treated.

5. The method of claim 1, wherein the effective amount is about 25 micrograms per kilogram to about 50 micrograms per kilogram of the subject being treated.

6. The method of claim 1, wherein the effective amount is about 50 micrograms per kilogram of the subject being treated.

7. The method of claim 1, wherein the surgery is selected from the group consisting of abdominopelvic surgery, abdominal surgery, pelvic surgery, cardiac surgery, and thoracic surgery.

8. The method of claim 1, wherein the subject exhibits reduced symptoms of post-post-surgical adhesions.

9. The method of claim 1, wherein the subject exhibits reduced indicators of post-surgical inflammation as measured in the surgical body cavity fluid.

10. The method of claim 9, wherein reduced indicators of post-surgical inflammation are selected from the group consisting of II-6, TNFα, TGFβ and IFN-γ.

11. The method of claim 1 wherein the subject exhibits increased levels of tPA in the surgical body cavity fluid.

12. The method of claim 1 wherein the variant of activated protein C consist of SEQ ID NO:1, containing conservative amino acid substitutes, secreted from an eukaryotic cell, and activated in vitro, wherein the activated protein C comprises cytoprotective activity.

13. The method of claim 1 wherein the variant of activated protein C consist of SEQ ID NO:2, containing conservative amino acid substitutes, secreted from an eukaryotic cell, and activated in vitro, wherein the variant of activated protein C comprises cytoprotective activity and reduced anticoagulant activity.

14. The method of claim 1 wherein the subject is a human subject.

15. The method of claim 1 wherein the subject is a non-human mammalian subject.

16. A method of reducing the incidence of post-surgical adhesions in a human subject undergoing abdominal surgery, the method comprising,
a) completing a primary surgery,
b) topically administering about 50 micrograms per kilogram of the human subject, of a variant of activated protein C comprising cytoprotective activity and reduced anticoagulant activity, to the organs and tissues exposed by the primary surgery or subjected to surgical manipulation by the primary surgery, and
c) closing a major incision associated with the primary surgery.

17. The method of claim 16, wherein the variant of activated protein C consists of the polypeptide set forth in SEQ ID NO: 2 with conservative amino acid substitutes, secreted from a eukaryotic cell, and activated in vitro, wherein the variant of activated protein C comprises cytoprotective activity and reduced anticoagulant activity.

18. The method of claim 16, wherein the variant of activated protein C consists of the polypeptide set forth in SEQ ID NO: 2 secreted from a eukaryotic cell, and activated in vitro.

19. The method of claim 16, wherein the surgery is selected from the group consisting of abdominopelvic surgery, abdominal surgery, pelvic surgery, cardiac surgery, and thoracic surgery.

20. The method of claim 16, wherein the subject is a human subject.


SEQUENCE LISTING (8 sequences)

SEQ ID NO: 1 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 2 - protein, 461 aa, Homo sapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVCLGEYDLRRWEKWELCLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - DNA, 33 bp, Homo sapiens
aagaagctccttgtctgccttggagagtatgac

SEQ ID NO: 4 - DNA, 33 bp, Homo sapiens
gtcatactctccaaggcagacaaggagcttctt

SEQ ID NO: 5 - DNA, 33 bp, Homo sapiens
gagaagtgggagctgtgcctggacatcaaggag

SEQ ID NO: 6 - DNA, 33 bp, Homo sapiens
ctccttgatgtccaggcacagctcccacttctc

SEQ ID NO: 7 - DNA, 1776 bp, Homo sapiens
gctgtcatggcggcaggacggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaaaaaaaaaaaaaaaaaaaaaaa

SEQ ID NO: 8 - DNA, 1776 bp, Homo sapiens
gctgtcatggcggcaggacggcgaacttgcagtatctccacgacccgcccctacaggtgccagtgcctccagaatgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtctgccttggagagtatgacctgcggcgctgggagaagtgggagctgtgcctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttagcgaccctccctgcagggctgggcttttgcatggcaatggatgggacattaaagggacatgtaacaagcacaccggcctgctgttctgtccttccatccctcttttgggctcttctggagggaagtaacatttactgagcacctgttgtatgtcacatgccttatgaatagaatcttaactcctagagcaactctgtggggtggggaggagcagatccaagttttgcggggtctaaagctgtgtgtgttgagggggatactctgtttatgaaaaagaataaaaaacacaaccacgaaaaaaaaaaaaaaaaaaaaaaa